(19) United States
(12) Patent Application Publication
(10) Pub. No.: US 2015/0150646 A1
(43) Pub. Date: Jun. 4, 2015

(54) AUTOCLAVABLE INPUT DEVICES

(71) Applicants: Timothy Pryor, Oakville (CA); Tyler David Ackland, Hamilton (CA)

(72) Inventors: Timothy Pryor, Oakville (CA); Tyler David Ackland, Hamilton (CA)

(21) Appl. No.: 14/536,403

(22) Filed: Nov. 7, 2014

Related U.S. Application Data
(63) Continuation-in-part of application No. 14/319,662, filed on Jun. 30, 2014.
(60) Provisional application No. 61/901,202, filed on Nov. 7, 2013.

(51) Int. Cl. A61B 19/00; G06F 3/044; G06F 3/039; G06F 3/0362

(52) U.S. Cl. CPC A61B 19/56; G06F 3/0362; G06F 3/044; G06F 3/039; A61B 2019/4868

(57) ABSTRACT

One embodiment describes a handheld, sterile input device to control one or more devices in the operating room. The embodiment's disposable component contains no electronics while its removable sensing module can be autoclaved and recharged for multiple procedures. Another embodiment describes a fully autoclaveable sterile input device with a detachable control assembly that enables thorough cleaning and disinfecting prior to steam sterilization in an autoclave.

### BACKGROUND OF THE INVENTION

Surgeons rely on medical imaging and other digital data to make informed decisions during a procedure. However, common computer peripherals such as the keyboard and mouse are non-sterile, making them difficult for a surgeon to use when scrubbed in and in the sterile field. Furthermore, an increasing number of surgeons store imaging and case data on their mobile devices; the sterile barrier, however, has limited their use during a procedure.

Surgeons and other members of the scrubbed-in surgical team want to directly control digital data and equipment while in the sterile field, without moving away from the operating table and without relying on non-sterile assistants to perform these tasks. A need exists, therefore, for an intuitive sterile input device, usable by surgeons and their teams within the sterile field.

Autoclaves are a preferred method of sterilizing surgical instruments at the hospital. After a procedure, many surgical instruments are steam-sterilized in an autoclave in preparation for a future procedure. Traditional input devices cannot be sterilized in an autoclaved, as doing so would render the devices useless.

Therefore, an input device that could be sterilized in an autoclave would fit within existing hospital workflows while providing many benefits to surgeons and their teams during a procedure.

### SUMMARY

Disclosed herein are various embodiments of a sterile input device for use in operating rooms, interventional radiology suites and other environments where the practitioner must maintain sterility when accessing medical equipment, medical imaging, mobile device applications and other digital data.

Some embodiments feature an autoclavable sensing module that is inserted into a sterile housing prior to a procedure. This allows members of the scrubbed-in, surgical team to directly handle the module when preparing for a surgical procedure.

Another embodiment features a fully autoclavable sterile controller, with an easy-to-clean detachable control assembly. It can be recharged while maintaining device sterility and can be manipulated by members of the scrubbed-in surgical team.

To better understand the nature and advantages of the present invention, reference should be made to the following description and the accompanying figures. It is to be understood, however, that each of the figures is provided for the purpose of illustration only and is not intended as a definition of the limits of the scope of the present invention.

### BRIEF DESCRIPTION OF THE DRAWINGS

FIG. 1 shows an embodiment of a sterile controller held in a user's hand.

FIG. 2A shows the components of an embodiment of a sterile controller.

FIG. 2B shows a sensing module contained in a sterile housing along with a sterile control assembly from one embodiment of a sterile controller.

FIG. 2C shows an embodiment of a fully assembled sterile controller.

FIG. 3A shows the components of an example configuration of a sterile controller.

FIG. 3B shows the components of an example configuration of an assembled sterile controller.

FIG. 4A shows a side view of one embodiment of a sterile control assembly and sterile housing contained in sterile packaging.

FIG. 4B shows a side view of one embodiment of a sensing module being inserted into a sterile housing.

FIG. 4C shows one embodiment of a sterile controller, with the sensing module enclosed by a sterile control assembly fastened to the sterile housing of FIG. 4B.

FIG. 5A shows an embodiment of an autoclavable sensing module for use in a sterile controller.

FIG. 5B shows the autoclavable sensing module from FIG. 5A being charged on an inductive charging base station.

FIG. 6A shows a camera and computer vision-based embodiment of an autoclavable sensing module.

FIG. 6B shows the autoclavable sensing module from FIG. 6A being charged on an inductive charging base station.

FIG. 7A shows the components of an example configuration of a sterile controller that uses an autoclavable sensing module

FIG. 7B shows the components of an example configuration of an assembled sterile controller that uses an autoclavable sensing module.

FIG. 8A shows the rear-view of an embodiment of a sterile control assembly for use with a camera and computer vision-based sterile controller system.

FIG. 8B shows the embodiment of FIG. 8A after the multifunctional controller knob has been turned 90 degrees.

FIG. 8C shows the embodiment of FIG. 8A after a push-switch has been pressed.

FIG. 9A shows a side view of an embodiment of an autoclavable sensing module employing a capacitive sensor.

FIG. 9B shows a side view of an embodiment of an assembled sterile controller that uses the autoclavable sensing module of FIG. 9A.

FIG. 10A shows a side view of an embodiment of a multifunctional controller knob for use with the autoclavable sensing module of FIG. 9A.

FIG. 10B shows a rear view of an embodiment of a multifunctional controller knob for use with the autoclavable sensing module of FIG. 9A.

FIG. 10C shows the directions the multifunctional controller knob embodiment of FIG. 9B can move in the x and y planes.

FIG. 11A shows embodiments of a sterile control assembly, sterile housing and autoclavable sensing module employing a contactless capacitive sensor.

FIG. 11 B shows an embodiment of an assembled sterile controller employing the autoclavable sensing module shown in FIG. 11 A.

FIG. 12 is a flow diagram detailing the creation of an embodiment of a sterile controller using an autoclavable sensing module.

FIG. 13A shows an example configuration of a fully autoclavable sterile controller system with a detached control assembly.

FIG. 13B shows an example configuration of an assembled, fully autoclavable sterile controller system being charged on an inductive charger.

FIG. 14 is a flow diagram detailing the creation of an embodiment of a fully autoclavable sterile controller.

### DETAILED DESCRIPTION

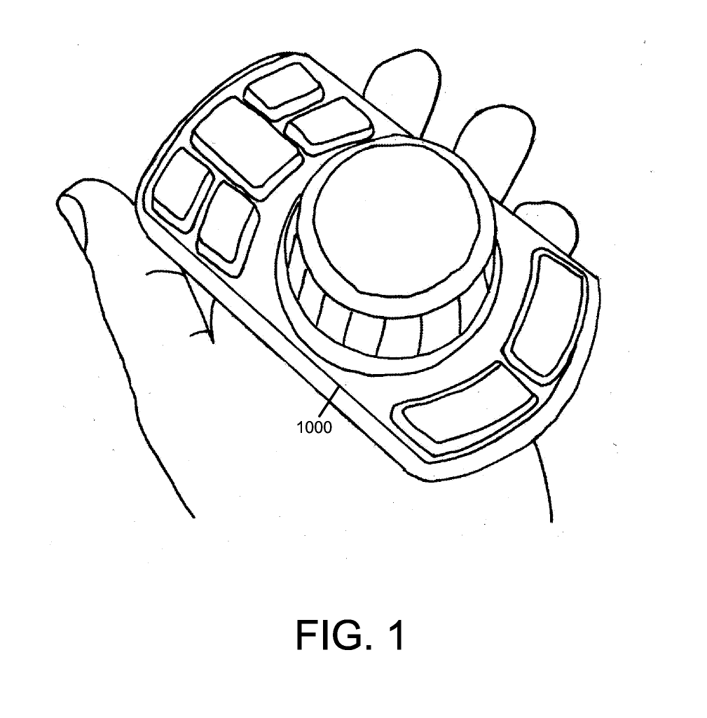

FIG. 1 shows a handheld embodiment of sterile controller 1000. It is a sterile, handheld controller that can be used within the sterile field during a procedure, to control a multitude of medical devices, computer equipment, mobile device data and other functionality in the operating room.

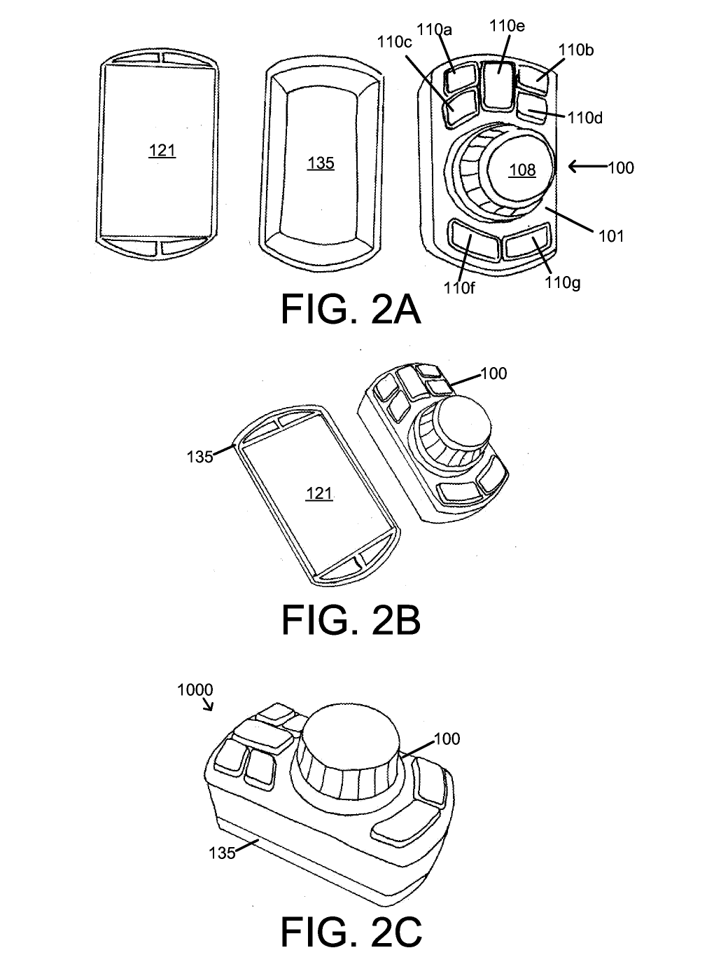

FIG. 2A shows the various components of the embodiment shown in FIG. 1. On the right of FIG. 2A, sterile control assembly 100 is shown. Sterile control assembly 100 includes push-switches 110a, 110b, 110c, 110d, 110e, 110f, 110g and multifunctional controller knob 108. These physical controls are mounted to sterile faceplate 101. These physical controls, mounted to sterile faceplate 101, form sterile control assembly 100.

Sterile housing 135 is shown in the center of FIG. 2A. Sensing module 121 is shown on the left. In this embodiment sensing module 121 is non-sterile. Sensing module 121 provides both the power for the device and registers physical control inputs. In this embodiment it is recharged before a procedure.

FIG. 2B shows sensing module 121 placed in sterile housing 135. By inserting sensing module 121 into sterile housing 135, sensing module 121's non-sterile, outer surfaces are protected by sterile housing 135's sterile outer surfaces. At this point, only sensing module 121's front face is exposed.

FIG. 2C shows sterile control assembly 100 fastened to sterile housing 135. This completely encloses the sensing module, forming sterile controller 1000. All outside surfaces are sterile to the touch, regardless of whether the sensing module is sterile or not. This allows the surgeon to use the controller within the sterile field without risk of contamination.

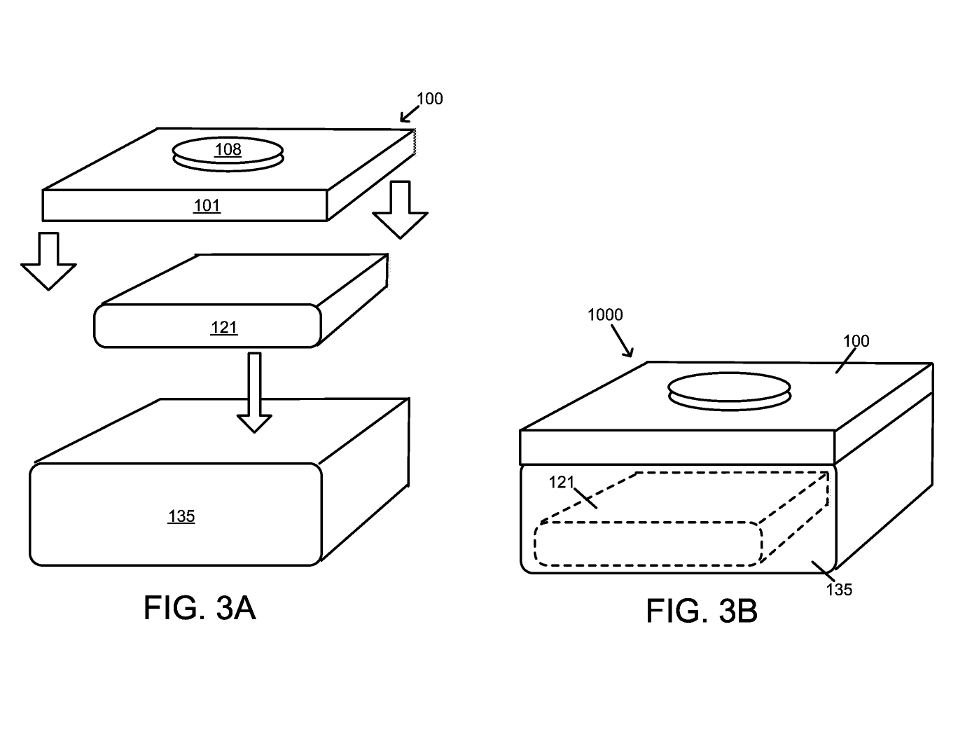

FIGS. 3A and 3B show a perspective view of an embodiment of the sterile controller concept. To create a sterile controller, sensing module 121 is inserted into sterile housing 135 (FIGS. 3A and 3B). Sterile control assembly 100 is then fastened to sterile housing 135 (FIG. 3B), leaving sensing module 121 completely enclosed by sterile control assembly 100 and sterile housing 135. In this embodiment, multifunctional controller knob 108 is the only control mounted to sterile control assembly 100. Sensing module 121 can sense the multifunctional controller knob 108 and send control state information to an external computer via a wireless transmitter.

The outside of both sterile control assembly 100 and sterile housing 135 are sterile to the touch, enabling sterile controller 1000 to be used in the sterile field, during a procedure.

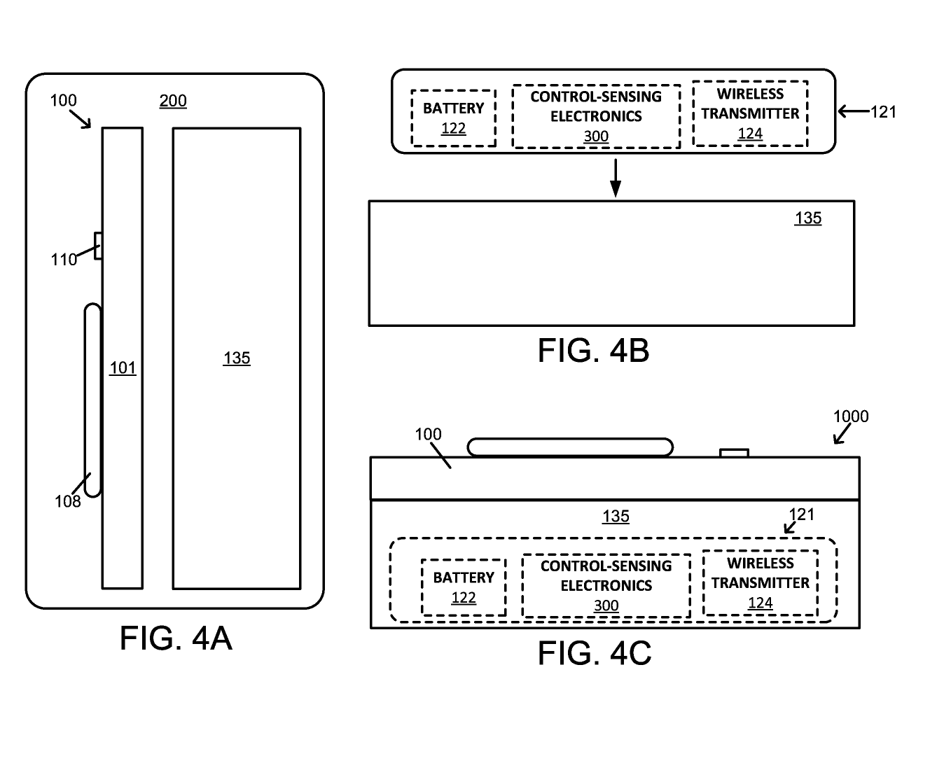

FIGS. 4A to 4C show a side view of an example configuration of the sterile controller system.

FIG. 4A shows disposable sterile housing 135 and sterile control assembly 100. In this embodiment, sterile control assembly 100 is made up of a multifunctional controller knob 108 and push-switch 110 which are mounted to sterile faceplate 101. Sterile housing 135 and sterile control assembly 100 are packaged in sterile packaging 200.

Sensing module 121 contains battery 122, wireless transmitter 124 and control-sensing electronics 300. Control-sensing electronics can refer to any method of sensing control state information. In this embodiment, controls include a multifunctional controller knob 108 and a push-switch 110. In this embodiment, sensing module 121 is non-sterile.

In the operating room, sterile packaging 200 is opened, and the sterile components are removed by a sterile member of the operating team. Sensing module 121 is inserted into sterile housing 135, as shown in FIG. 4B and FIG. 4C. Sterile controller 1000 is created when a scrubbed-in member of the operating team fastens sterile control assembly to sterile housing 135, completely enclosing sensing module 121 (FIG. 4C). In this way, all outside surfaces are sterile to the touch, whether sensing module 121 is sterile or not. This enables the electronics to be reused over multiple procedures, instead of being discarded, as is common with sterile, disposable electronics. It also enables the sterile control assembly 100 and sterile housing 135 to be very cost-effective and environmentally-friendly when compared to comparable disposable electronics devices. In other disposable electronics devices, the electronics are disposed of every procedure, as opposed to being reused, as shown in FIGS. 4A to 4C.

As the surgeon or other practitioner changes the state of the multifunctional controller knob 108 or push-switch 110, control-sensing electronics 300 registers these changes and transmits control state information via wireless transmitter 124.

In the embodiments shown in FIGS. 2A, 2B, 2C, 3A, 3B, 4A, 4B and 4C, sensing module 121 is non-sterile and can be recharged before a procedure in the operating room. By placing it in sterile housing 135, and covering it with sterile control assembly 100, the non-sterile sensing module 121 cannot be touched by a member of the scrubbed-in, surgical team. Below we discuss ways in which the sensing module could be sterilized before being placed in the sterile housing.

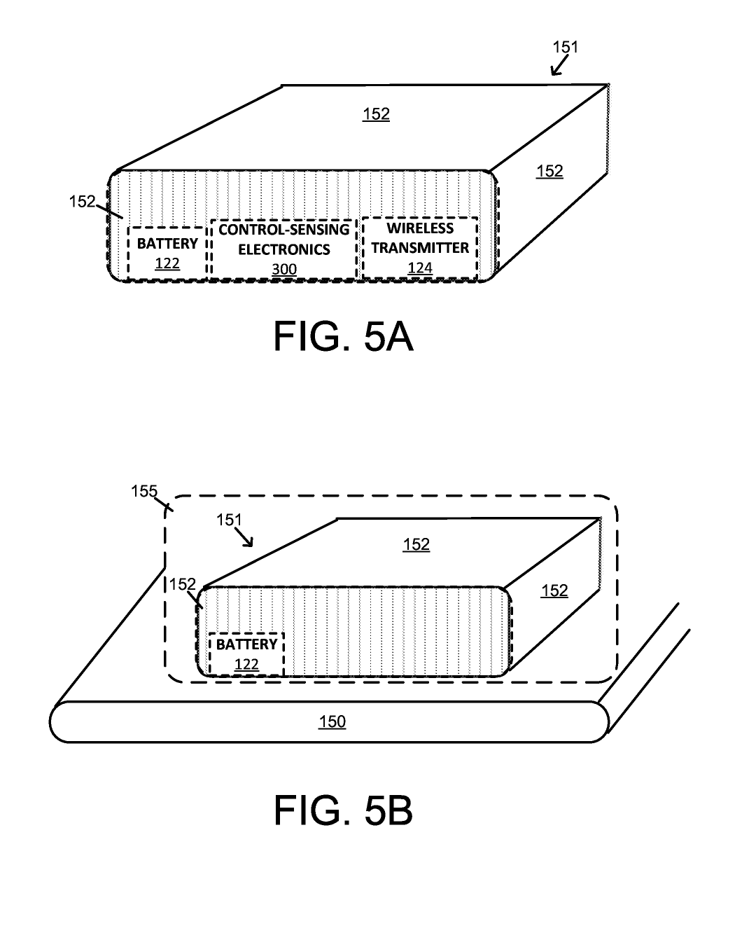

FIGS. 5A and 5B show an example configuration of an autoclavable sensing module. In the hospital, steam sterilization autoclaves are a preferred method for sterilizing reusable surgical instruments. Autoclave temperatures can reach as high as 148 degrees Celsius and generate as much as 30 PSI of pressure. Traditional user interfaces cannot be autoclaved due to these conditions.

As shown in FIG. 5A, the electronics of the autoclavable sensing module 151 could withstand an autoclave's environmental conditions. FIG. 5A shows autoclavable sensing module 151, which consists of battery 122, control sensing electronics 300 and wireless transmitter 124. Thermal insulating material 152 protects the various electronics from the overheating that could occur due to the conduction that would result when autoclavable sensing module 151 is placed in an autoclave. For example, conventional flash memory would lose its programmed contents and conventional batteries would lose capacity or be destroyed in such conditions.

In FIG. 5A, thermal insulating material 152 (represented by the bars on the front of autoclavable sensing module 151 in FIG. 5A) lines the interior walls of autoclavable sensing module 151. This insulates the interior of autoclavable sensing module 151, and protects battery 122, control sensing electronics 300 and wireless transmitter 124 from overheating while in the autoclave.

The autoclavable sensing module 151 can be made of an autoclavable material such as stainless steel, glass or suitable thermoplastic and can be completely sealed to withstand steam ingress at the pressures generated by a typical autoclave.

As an alternative to the use of a thermal insulating material, a partial vacuum could be created within autoclavable sensing module 151 using various methods. For instance two halves of the sensing module could be joined with a gasket within a vacuum environment. Or, a valve could be integrated into the sensing module for drawing a vacuum after assembly. This valve could also be used to recreate the partial vacuum if the electronics in the autoclavable sensing module needed servicing. The partial vacuum created within the sensing module would keep the electronics protected from the extreme conditions in the autoclave, as less air molecules are available to collide and conduct heat.

Prior to being placed in the autoclave, autoclavable sensing module 151 is placed in an autoclave bag 155. The bagged autoclavable sensing module 151 is then placed in an autoclave for sterilization. Upon completion of the sterilization cycle, it is removed from the autoclave, and autoclave bag 155 is sealed. FIG. 5B shows a bagged autoclavable sensing module 151 placed on inductive charging base station 150. After being sterilized, the autoclavable sensing module can be recharged via inductive charging. By remaining in autoclave bag 155, autoclavable sensing module 151 maintains its sterility, even as it is placed on inductive charging base station 150. Battery 122 includes appropriate charge regulation circuitry and an electromagnetic coil to enable inductive charging.

Some newer electronics can be designed to withstand autoclave conditions, such as autoclavable batteries and autoclavable flash memory. The autoclavable module shown in FIGS. 5A and 5B could be designed without an insulating barrier or without creating a partial vacuum in the module, if the module were composed of suitable autoclavable electronic components.

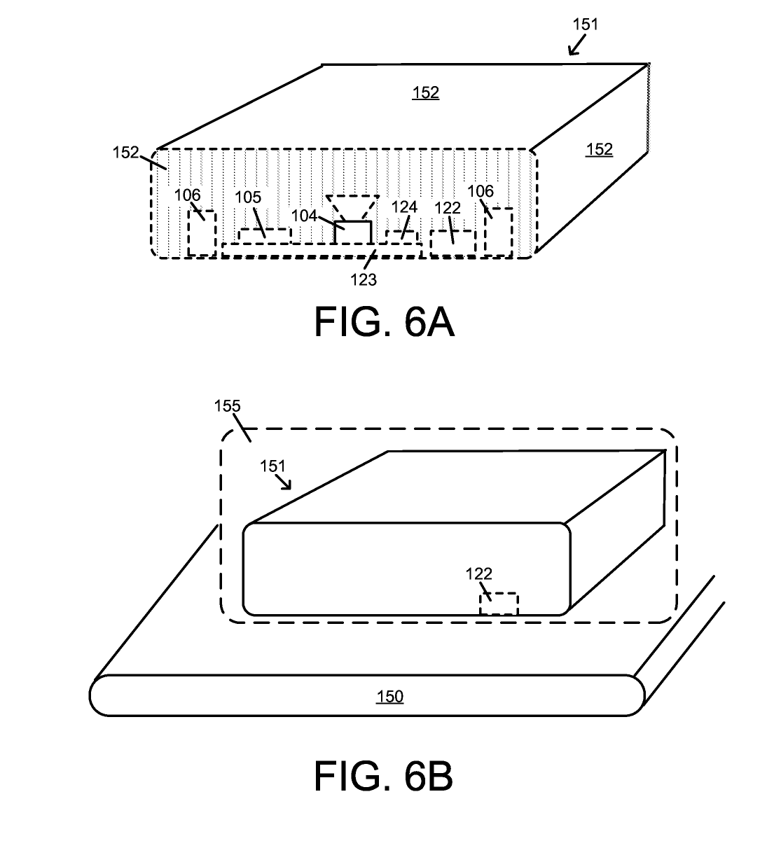

FIGS. 6A and 6B show an example configuration of an autoclavable sensing module 151 that uses a camera and computer vision system. In FIG. 6A, autoclavable sensing module 151 contains camera 104, LEDs 106, processor 105, motherboard 123, battery 122 and wireless transmitter 124. Thermal insulating material 152 protects the various electronics from damage due to heat conducting into the sensing module from the autoclave.

The camera and computer vision system sense control position locations, and transmit this information via wireless transmitter 124. The top of autoclavable sensing module 151 could be created out of a transparent autoclavable material such as glass in order that the camera could see the targets located on the rear of the physical controls. A void in the insulating material would be required so as not to block the camera's field of view.

FIG. 6B shows the autoclavable sensing module 151 described in FIG. 6A placed on inductive charging base station 150, which charges battery 122. Sterilized sensing module 151 can maintain its sterility by remaining in autoclave bag 155 while placed on non-sterile inductive charging base station 151. Battery 122 includes appropriate charge regulation circuitry and an electromagnetic coil to enable inductive charging.

As an alternative to the use of a thermal insulating material, a partial vacuum could be created within autoclavable sensing module 151 using various methods. For instance two halves of the sensing module could be joined with a gasket within a vacuum environment. Or, a valve could be integrated into the sensing module for drawing a vacuum after assembly. This valve could also be used to recreate the partial vacuum if the electronics in the autoclavable sensing module needed servicing. The partial vacuum created within the sensing module would keep the electronics protected from the extreme conditions in the autoclave, as less air molecules are available to collide and conduct heat.

As an alternative to using a sterile housing, the camera and computer vision implementation of the autoclaveable sensing module could be inserted into a non-sterile housing, if such a housing were inserted inside a transparent sterile bag. The sterile control assembly could be attached to the housing, overtop of the sterile bag. The camera could detect control target positions through the bag.

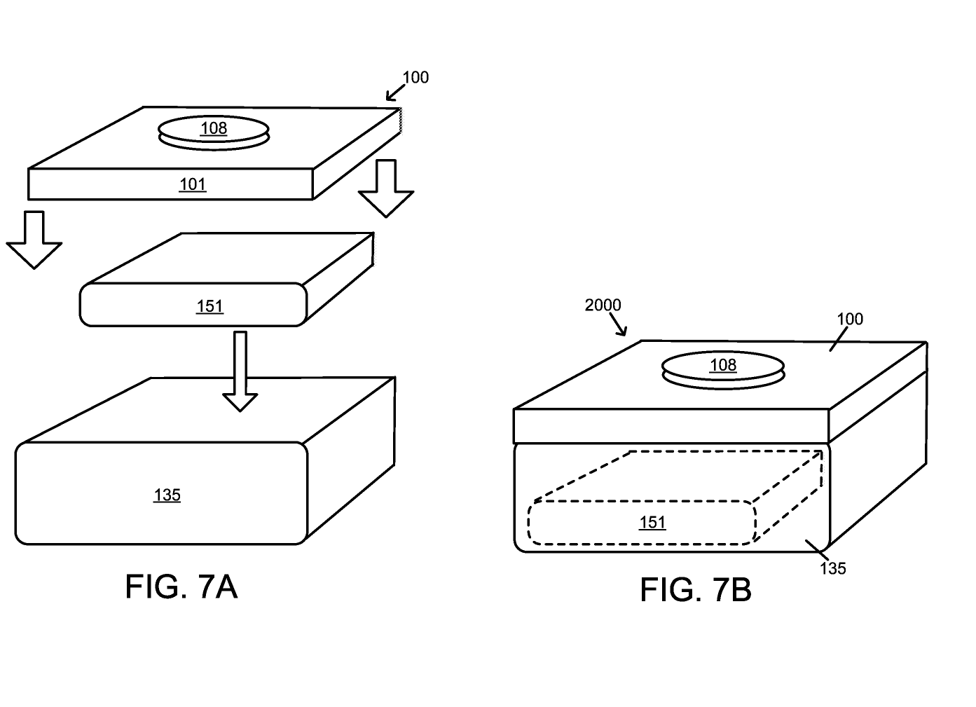

FIGS. 7A and 7B show a sterile controller system using autoclavable sensing module 151, after it has been sterilized in an autoclave. FIG. 7A shows the components of an embodiment of a sterile controller system, including sterile housing 135, autoclavable sensing module 151 and sterile control assembly 100. Sterile control assembly 100 is composed of multifunctional control knob 108 mounted to sterile faceplate 101.

FIG. 7B shows autoclavable sensing module 151 placed inside sterile housing 135 and covered with sterile control assembly 100, creating sterile controller 2000. Autoclavable sensing module 151 senses multifunctional knob 108's position and transmits this information via its wireless transmitter. Given that autoclavable sensing module 151 is sterile, no special handling precautions need to be taken when placing it in sterile housing 135.

Sterile housing 135 and sterile control assembly 100 can be delivered to the operating room in sterile packaging and can be disposed of after the procedure. Alternatively, sterile housing 135 and sterile control assembly 100 could be constructed of materials suitable for sterilization in an autoclave or gas plasma sterilization machine. They could then be re-sterilized after the procedure.

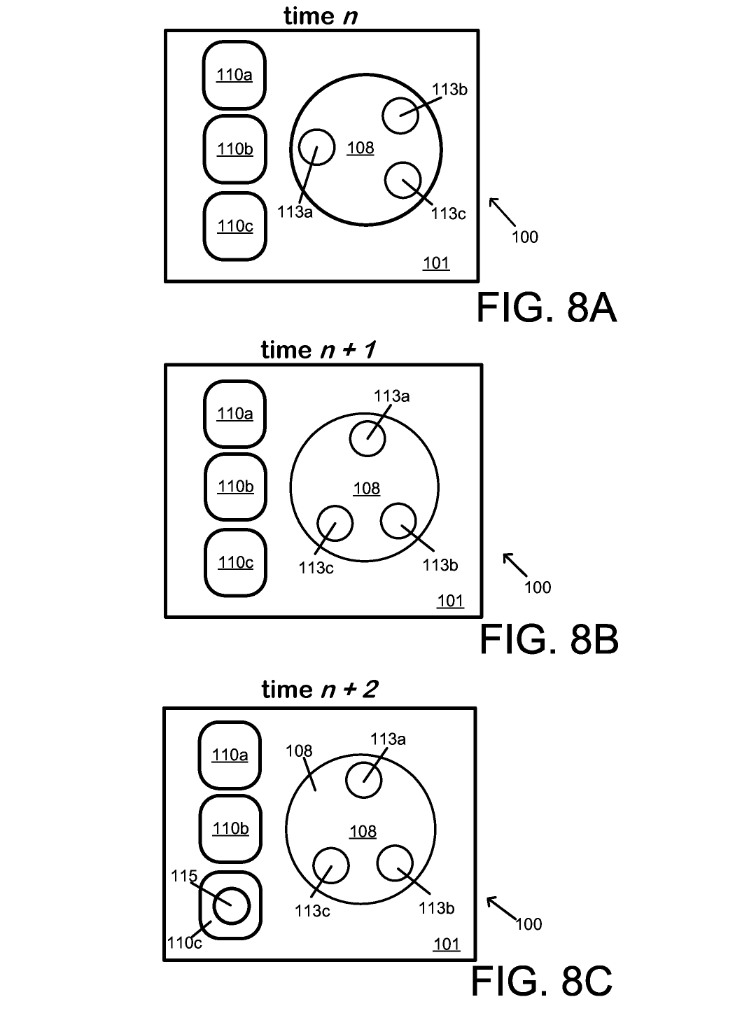

FIGS. 8A to 8C show how the physical control targets in a camera-based implementation of the sterile controller can change over time. FIGS. 8A to 8C show the rear of a sterile control assembly 100, which consists of a multifunctional controller knob 108 and push-switches 110a, 110b and 110c mounted to sterile faceplate 101. X-Y plane targets 113 are affixed to multifunctional control knob 108. At time n (FIG. 8A), push-switches 110a, 110b, 110c have not been pressed by the user. At time n+1 (FIG. 8B), multifunctional controller knob 108 has rotated 90 degrees in the clockwise direction. X-Y plane targets 113a, 113b, 113c have rotated with multifunctional controller knob 108. The camera and processor register this change, and transmit the state change information via the wireless transmitter. At time n push-switches 110a, 110b, 110c still haven't been pressed by the user. At time n+2 (FIG. 8C), push-switch 110c has been pushed by the user, revealing Z-plane target 115 to the camera. The camera and processor would register this change and transmit the state change information via the wireless transmitter. At time n+2, multifunctional controller 108 and its associated X-Y plane targets 113a, 113b and 113c have not rotated further, and push-switches 110a and 110b are not being pushed.

Note that other target designs are possible with the controls shown in FIGS. 8A to 8C. For instance, with the Z-plane push-switch targets, a partial target could be showing when the push-switch was not activated. When the push-switch is activated, it could increase in size, and this could be registered as an input.

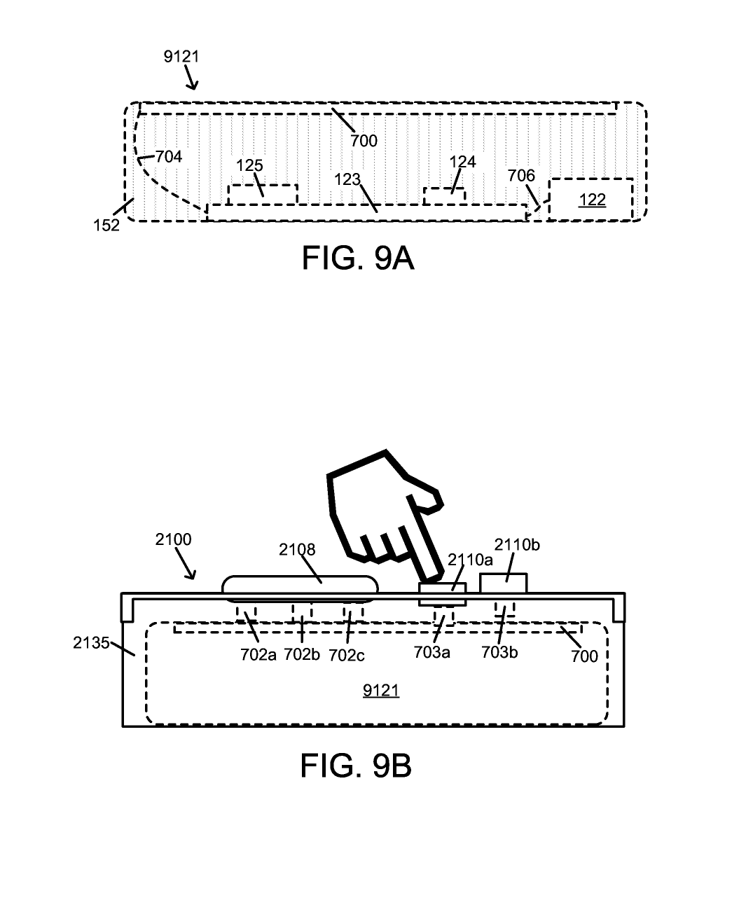

FIGS. 9A and 9B show a capacitive sensor implementation of an autoclavable sensing module. In FIG. 9A, processor 125 and wireless transmitter 124 are mounted to motherboard 123. Battery 122 powers autoclavable sensing module 9121 and is connected to motherboard 123 using battery cable 706. Capacitive sensor 700 is placed at the top of sensing module 151 and is connected to the motherboard via flat flex cable 704.

Thermal insulating material 152 (represented by the bars on the front of autoclavable sensing module 151 in FIG. 9A) lines the interior walls of autoclavable sensing module 151. This insulates the interior of autoclavable sensing module 151 and protects the various electronic components from damage due to overheating while in the autoclave.

As an alternative to the use of a thermal insulating material, a partial vacuum could be created within autoclavable sensing module 9121 using various methods. For instance two halves of the sensing module could be joined with a gasket within a vacuum environment. Or, a valve could be integrated into the sensing module for drawing a vacuum after assembly. This valve could also be used to recreate the partial vacuum if the electronics in the autoclavable sensing module needed servicing. The partial vacuum created within the sensing module would keep the electronics protected from the extreme conditions in the autoclave, as less air molecules are available to collide and conduct heat.

In FIG. 9B, autoclavable sensing module 9121 is placed in sterile housing 2135, along with a sterile control assembly 2100 designed for use with the capacitive sensor implementation. Multifunctional controller knob 2108 includes electrically conductive targets 702a, 702b, 702c, and push-switches 2110a and 2110b include conductive targets 703a and 703b, respectively. Multifunctional controller knob 2108 and push-switches 2110a and 2110b are mounted to sterile faceplate 2101, which form sterile control assembly 2100, designed for use in a capacitive implementation of the sterile controller. The conductive targets can be sensed by a capacitive sensor.

Modern smartphones and tablets are often supplied with capacitive touch screens that can sense an object such as a conductive stylus. The Samsung Galaxy Note, for example, provides a stylus made of conductive material and can register a user's handwriting using the stylus. The Note can also register multi-touch from the user's fingers, which are themselves conductive objects. In the same way, the conductive targets 702a, 702b, 702c, 703a, 703b can be made of similar material to the Note's stylus, or of other conductive material, and can be sensed by a conductive sensor. The targets could be made out of a conductive material such as graphite in order to be sensed by the capacitive sensor. A capacitive sensor can sense conductive material as it touches the sensor. The capacitive sensor can also sense the conductive material if the material is located slightly above, but not touching, the sensor.

FIG. 9B shows a sterile controller, composed of the capacitive autoclavable sensing module 9121 and enclosed by sterile housing 2135 and sterile control assembly 2100. When sterile control assembly 2100 is fastened to sterile housing 2135, conductive knob targets 702a, 702b, 702c (affixed to multifunctional controller knob 2108) touch the capacitive sensor 700, or are located slightly above capacitive sensor 700, close enough for the targets to be sensed. Conductive target 703a is attached to push-switch 2110a, and is being pushed by the user. Conductive target 703a touches capacitive sensor 700, which registers the location of the target. Alternatively, the system could be designed so that conductive target 703a could be sensed if it moved towards capacitive sensor 700, but did not touch it. As mentioned above, a capacitive sensor can sense a conductive target if it is located slightly above the capacitive sensor. Push-switch 2110b is not being pushed, and therefore its associated conductive target 703b is not close enough to conductive sensor 700 to be registered as an input.

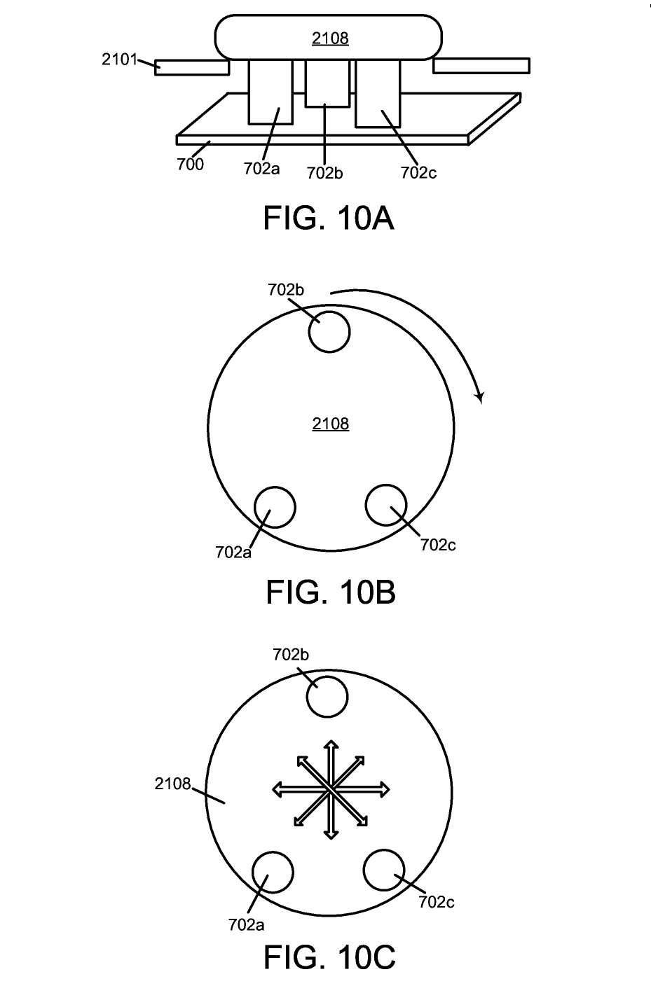

FIGS. 10A to 10C show how a capacitive sensor can sense conductive knob targets on a multifunctional controller knob.

FIG. 10A shows a side-view of multifunctional controller knob 2108 fitted with conductive targets 702a, 702b, 702c. Multifunctional controller knob 2108 is mounted on sterile faceplate 2101. When sterile faceplate 2101 is fastened on the sterile housing, the conductive targets come into contact with capacitive sensor 700, or are close enough to be sensed by the capacitive sensor. A capacitive sensor can sense several conductive targets simultaneously.

FIG. 10B shows a view of the rear of multifunctional controller knob 2108. Multifunctional controller knob 2108 rotates in a clockwise direction. Conductive sensor 700 (shown in FIG. 10A) can sense the rotation of three conductive targets 702a, 702b 702c simultaneously and determine the degree to which multifunctional controller knob 2108 was rotated by the user.

FIG. 10C also shows a view of the rear of multifunctional controller knob 2108. The arrows on multifunctional controller knob 2108 show how the controller knob can move in the manner of a joystick (up, down, left, right, diagonally). Conductive targets 702a, 702b, 702c are sensed as they move along the XY plane of the conductive sensor.

In FIGS. 10A to 10C, the targets are assumed to be in contact with the capacitive sensor or close enough to be sensed by the capacitive sensor.

Alternatively, a smaller number of targets could be used in conjunction with the multifunctional controller knob if the capacitive sensor can resolve the movements of the multifunctional controller knob associated with the smaller number of targets.

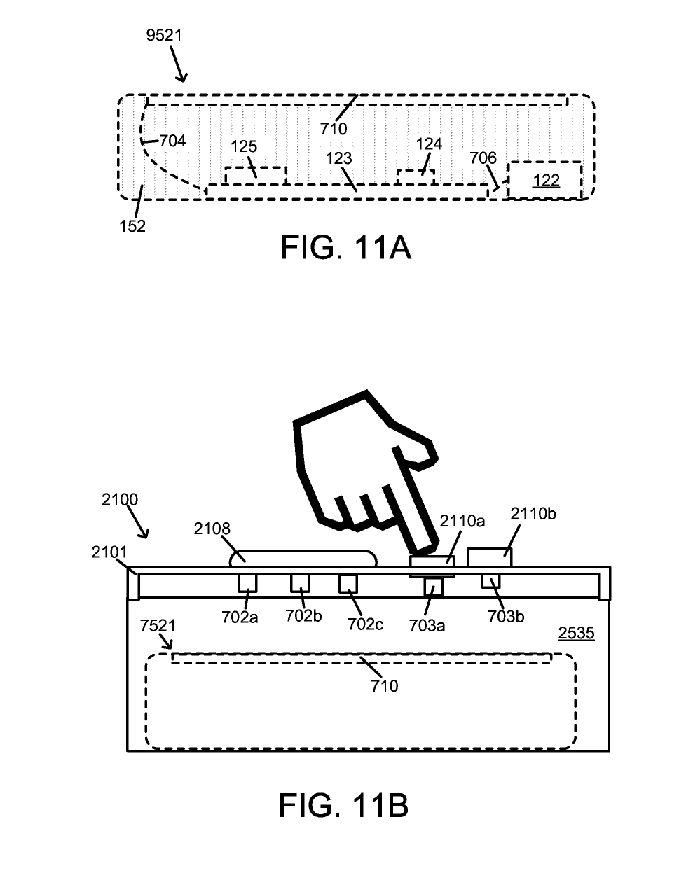

FIGS. 11A and 11B show an embodiment of the sterile controller using an autoclavable, contactless capacitive sensor. A contactless capacitive sensor 710, such as a sensor employing “Sensation” technology demonstrated by the Fogale Nanotech Corporation, can sense fingers and conductive objects up to a distance of 5 cm from the contactless capacitive sensor 710.

Autoclavable, conductive sensing module 9521 (FIG. 11A) is a variation on the sensing module shown in other embodiments. In this embodiment, autoclavable conductive sensing module 9521 is made of conductive materials, such as carbon filled acrylonitrile butadiene styrene.

In a similar manner, conductive sterile housing 2535 (FIG. 11 B) is a variation on the sterile housing shown in other embodiments. In this embodiment, conductive sterile housing 2535 is constructed out of conductive materials, such as carbon filled acrylonitrile butadiene styrene.

In the contactless capacitive sensor implementation shown in FIG. 11A, conductive targets 702a, 702b, 702c, 703a, 703b function as the opposing capacitor plate for this type of sensor, and the capacitance changes depending on the distance of the target to the contactless capacitive sensor 710.

A return path to contactless capacitive sensor 710 is required, and therefore the sterile housing 2535 and sensing module 9521 should be made of conductive material.

When the conductive targets 702a, 702b, 702c on multifunctional controller knob 2108 rotate, or the conductive targets 703a, 703b on push-switches 2110 move downwards, towards the sensor, the contactless capacitive sensor 710 can register these movements.

The autoclavable contactless capacitive sensing module 9521 shown in FIG. 11A is implemented using a processor 125 and wireless transmitter 124, both mounted to motherboard 123. Battery 122 powers the unit and is connected to motherboard 123 using battery cable 706. Contactless capacitive sensor 710 is placed at the top of autoclavable sensing module 9521, and is connected to the motherboard via flat flex cable 704

Thermal insulating material 152 (represented by the bars on the front of autoclavable sensing module 9521 in FIG. 11A) lines the walls of autoclavable sensing module 9521. This insulates the interior of autoclavable sensing module 9521, and protects the electronics from damage due to overheating while in the autoclave.

As an alternative to the use of a thermal insulating material, a partial vacuum could be created within autoclavable sensing module 9521 using various methods. For instance two halves of the sensing module could be joined with a gasket within a vacuum environment. Or, a valve could be integrated into the sensing module for drawing a vacuum after assembly. This valve could also be used to recreate the partial vacuum if the electronics in the autoclavable sensing module needed servicing. The vacuum created within the sensing module would keep the electronics protected from the extreme conditions in the autoclave, as less air molecules are available to collide and conduct heat.

FIG. 11 B shows the contactless capacitive sensor implementation of a sterile controller when autoclavable contactless capacitive sensing module 9521 has been placed in sterile housing 2535, and sterile control assembly 2100 has been fastened to sterile housing 2535. Sterile control assembly 2100 consists of multifunctional controller knob 2108, push-switch 2110a and push-switch 2110b mounted to sterile faceplate 2101. Note that when sterile control assembly 2100 has been mounted to sterile housing 2521, conductive targets 702a, 702b, 702c, 703a, 703b can be located at a distance of up to 5 cm from sensing module 9521, which consists of contactless capacitive sensor 710 and the components described in FIG. 11A.

In FIG. 11 B, the user is activating push-switch 2110a. Push-switch 2110a's associated conductive target 703a moves towards contactless conductive sensor 710 located within autoclavable sensing module 9521. Autoclavable sensing module 9521 registers this movement as an input and wirelessly transmits this input information.

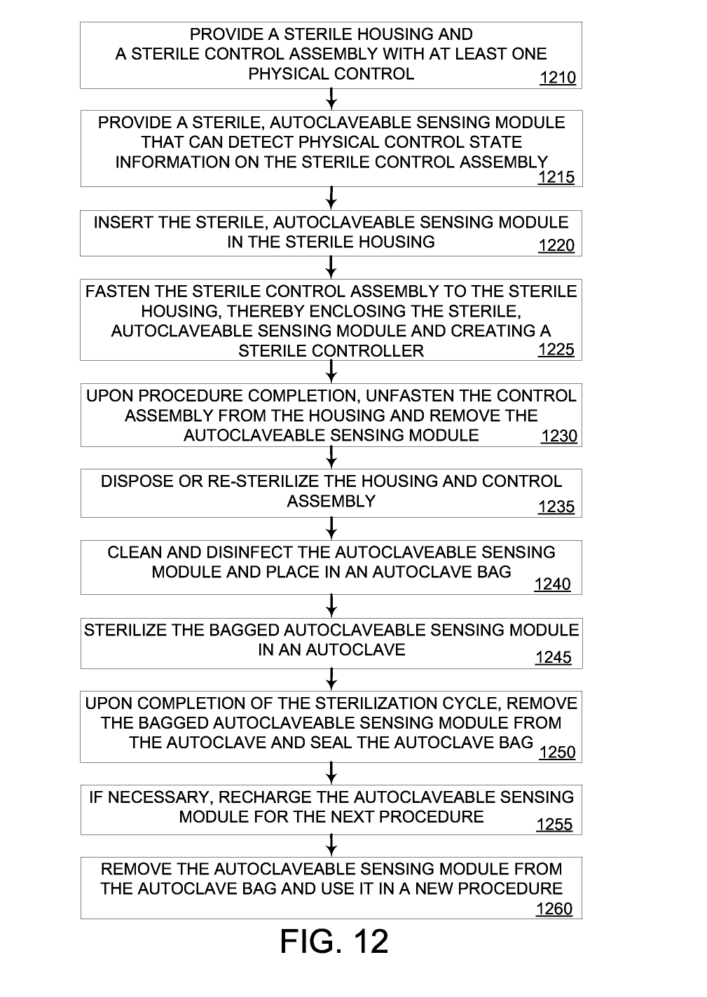

FIG. 12 is a flow diagram detailing the creation of a sterile controller system which uses an autoclavable sensing module. In step 1210, a sterile housing and sterile control assembly are provided. A sterile control assembly consists of one or more physical controls mounted to a sterile faceplate. The sterile housing and sterile control assembly could be delivered in sterile packaging from a sterilization facility, or could have been sterilized at the hospital, if a suitable material was chosen for the control assembly and housing.

In step 1215, an autoclavable sensing module is provided. The autoclavable sensing module can detect control state on one or more controls on the sterile control assembly. It is assumed that the autoclavable sensing module has been sterilized using an autoclave or other suitable sterilization method prior to the procedure. It is also assumed that it has been removed from the autoclave bag that was used to protect the module from contamination. In step 1220, the autoclavable sensing module is inserted in the sterile housing. In step 1225, the sterile control assembly is fastened to the sterile housing. This completely encloses the sensing module. All outside surfaces are sterile to the touch. In step 1230, the surgical procedure has been completed, and the housing and control assembly are now non-sterile. These are unfastened from each other, and the autoclavable sensing module is removed. In step 1235, the housing and control assembly are disposed of, or are re-sterilized. In step 1240, the autoclavable sensing module is cleaned and disinfected and placed in an autoclave bag. In step 1245 the bagged autoclavable sensing module is sterilized in an autoclave. In step 1250, the sterilization cycle is complete, and the bagged autoclavable sensing module is removed from the autoclave, and the autoclave bag is sealed to maintain module sterility. In step 1255, the autoclavable sensing module is recharged in preparation for the next procedure. In one embodiment the autoclavable sensing module could be recharged using inductive charging, as described in FIG. 3B. However, any recharging method that maintained the sterility of the autoclavable sensing module could be used. In step 1260, the autoclavable sensing module is removed from the autoclave bag in preparation for a new procedure.

The method described in FIG. 12 allows a scrubbed-in member of the surgical team to directly touch the autoclaveable sensing module prior to its insertion in the sterile housing. A non-sterile sensing module would require handling from a non-sterile assistant.

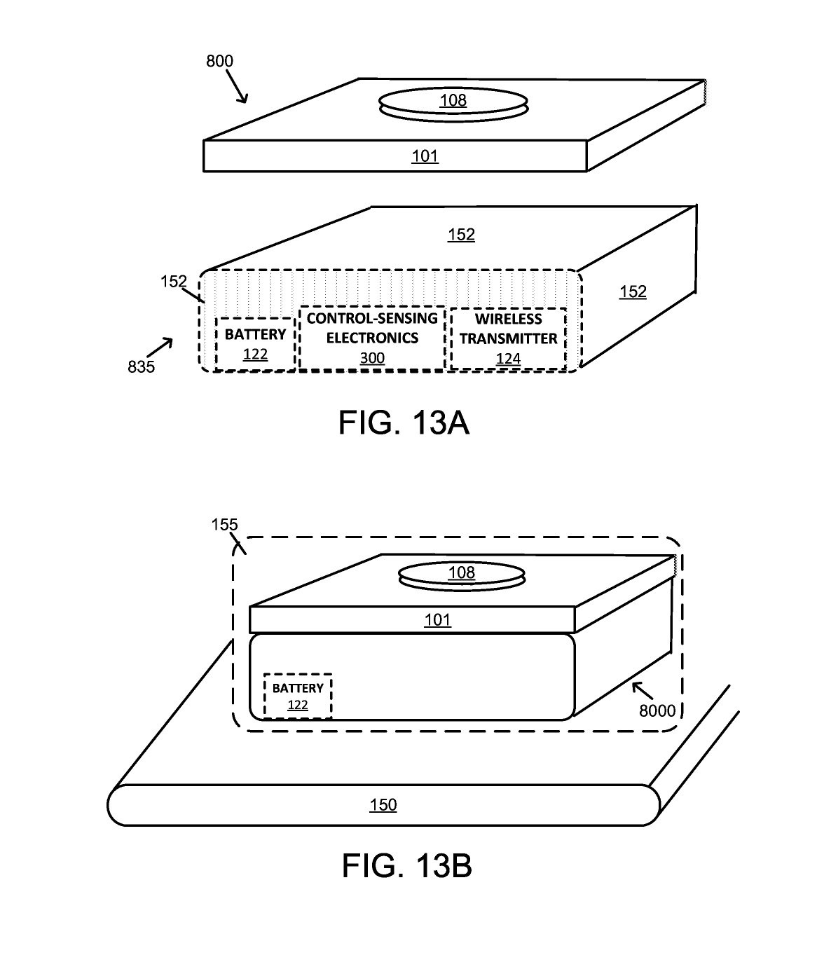

FIGS. 13A and 13B shows a fully autoclavable sterile controller featuring a detachable autoclavable control assembly.

The first step to properly sterilizing surgical instruments in a hospital often involves rinsing off blood, bodily fluids and tissue from the surgical instrument after a procedure. Then instruments are disinfected using an approved disinfectant. Once disinfected, instruments are typically cleaned using an enzymic cleaner bath or ultrasonic cleansing device. At this point, however, the surgical instrument is still not sterile. To ensure that the instrument is sterile, it is steam sterilized in an autoclave.

The embodiment shown in FIG. 13A enables an autoclavable input device for surgeons and features a detachable, autoclavable control assembly 800. This enables more thorough pre-washing, disinfecting and cleaning of the knobs, buttons and other physical controls that can be manipulated by a surgeon during a procedure.

Battery 122, control sensing electronics 300 and wireless transmitter 124 are included in sterile housing with electronics 835. In this embodiment, there is no separate sensing module, as shown in the embodiments above. Sterile housing with electronics 835 includes both the housing and the electronics necessary for sensing control state information and wirelessly transmitting such information. The outside of sterile housing with electronics 835 can be constructed of an autoclavable material such as autoclavable plastic or stainless steel while the interior electronics can be protected using thermal insulating material 152, or by creating a partial vacuum inside sterile housing with electronics 835. These techniques were discussed above in the context of an autoclavable sensing module. In FIG. 13A, thermal insulating material 152 lines sterile housing with electronics 835.

Autoclavable control assembly 800 can be unfastened from sterile housing with electronics 835 prior to autoclaving. This is advantageous in that during a procedure, a surgeon will handle the physical controls such as knobs and buttons. The controls could have blood, tissue and other debris residing within the control crevices. This would make these controls difficult to sterilize in an autoclave without first thoroughly pre-washing the controls. By removing the detachable, autoclavable control assembly 800, it can be more thoroughly washed, and debris such as blood and tissue can be removed more easily than if the control assembly were not removable.

If desired, a sterile, disposable control assembly could be used instead of autoclavable control assembly 800. This could be delivered to the operating room in sterile packaging, and fastened to the previously autoclaved sterile housing with electronics 835. Such an embodiment would avoid the cleaning and disinfecting of the detachable control assembly while still enabling the reuse of the electronics in multiple procedures.

The control sensing electronics can be implemented in a variety of ways. For example, a camera and computer vision system similar to that used by the autoclavable sensing module in FIG. 6A could be used. A capacitive sensor system similar to that used by the autoclavable sensing module in FIG. 9A could be used. A contactless capacitive sensor similar to that used by the autoclavable sensing module in FIG. 11A could also be used.

FIG. 13B shows the assembled autoclavable sterile controller 8000, once it has been steam-sterilized in an autoclave. It has been placed on inductive charging base station 150 and is being charged in preparation for a surgical procedure.

Battery 122 includes appropriate charge regulation circuitry and an electromagnetic coil to enable inductive charging. Alternatively, a properly designed sterile charging cable and battery could be used instead of the inductive charging system presented in FIG. 13B.

Prior to being placed in the autoclave, detachable control assembly 800 has been fastened to sterile housing with electronics 835, creating autoclavable sterile controller 8000. Autoclavable sterile controller 8000 is then placed in autoclave bag 155. The bagged autoclavable controller 8000 is then placed in the autoclave for sterilization. Upon completion of the sterilization cycle, it is removed from the autoclave, and autoclave bag 155 is sealed. The bagged autoclavable controller 8000 can then be placed on induction charging base station 150, as shown in FIG. 13B. This maintains the sterility of autoclavable controller 8000. When the procedure is set to begin, autoclavable controller is carefully removed from autoclave bag 155 to maintain the device's sterility.

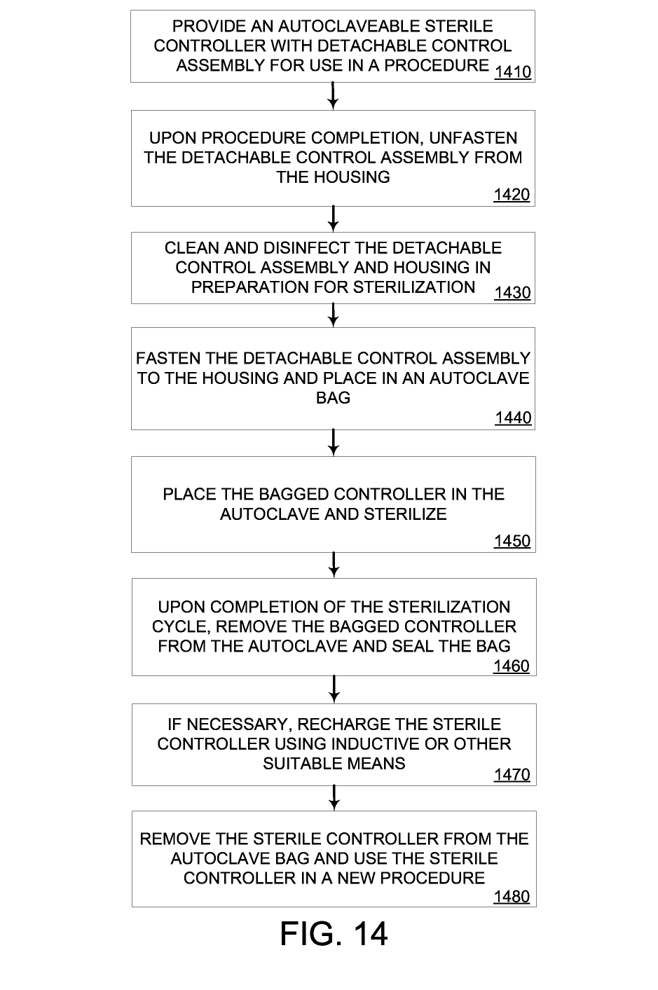

FIG. 14 is a flow diagram detailing the creation of an easily cleanable, fully autoclavable sterile controller system. In step 1410, a fully assembled and autoclavable sterile controller (such as described in FIGS. 13A and 13B) is provided for a procedure. Once the procedure is complete, the non-sterile detachable control assembly is unfastened from the sterile housing with electronics (step 1420). The detachable control assembly and sterile housing with electronics are then cleaned and disinfected, to remove any excess blood, tissue or other debris (step 1430). This will ensure proper sterilization. The detachable control assembly is then fastened to the housing, and the sterile controller is placed in an autoclave bag (step 1440). The bagged controller is then placed in the autoclave and sterilized (step 1450). Once the autoclave has completed its sterilization cycle, the detachable control assembly and sterile housing with electronics are removed from the autoclave and the autoclave bag is sealed (step 1460). If necessary, the sterile controller is recharged using inductive of other suitable means (step 1470). The sterile controller is removed from the autoclavable bag and can then be used in a new procedure (step 1480).

As noted above, the cleaning and disinfecting of the detachable control assembly (step 1430) could be avoided through the use of a sterile, disposable control assembly. This control assembly could be delivered to the operating room and opened prior to a procedure. It could then be fastened to the autoclaved sterile housing with electronics, creating a sterile input device for use in the sterile field.

### CONCLUSION, RAMIFICATIONS AND SCOPE

Accordingly, the reader will see that the input devices outlined in the various embodiments provide intuitive control over digital data and medical equipment from within the sterile field. Surgeons and other members of the scrubbed-in surgical team can use the input devices described above during a procedure. This streamlines surgical workflow and reduces overall procedure time and decreases the probability for errors.

Other types of control-sensing electronics could be used in both the autoclavable sensing module (FIG. 5A) and the fully autoclavable sterile controller (FIG. 13A). For example, a Hall-Effect sensing system could be used to determine control position locations if these controls were outfitted with suitable magnetic targets.

As another example, an infrared “light-grid” bezel could be used to sense control position locations if these controls were outfitted with suitable targets that crossed the plane of the bezel.

As another example, a non-contact inductive positional sensor could be used to determine rotational and linear positions of various controls, as long as these controls were outfitted with a metallic target, or activator, that would allow the sensor to determine angular or linear position.

As another example, an ultrasonic or laser time-of-flight sensor could be used to determine the positions of the various controls mounted to a sterile faceplate, as long as these controls were outfitted with suitable targets for sound or light reflection back to the sensor.

A resistive touch screen could be used as the sensor if controls were outfitted with targets that, when activated, applied pressure to the resistive touch screen.

As an alternative to electrochemical batteries, a supercapacitor and appropriate regulation circuitry could be used to power the sensing module.

As an alternative to the inductive charging method outlined in the description, a properly designed sterile charging cable could be used to recharge the device.

Ethylene oxide or vaporized hydrogen peroxide sterilization methods may be used instead of steam autoclaving if appropriate materials are selected to construct the controller components. These sterilization methods are inherently safe for enclosed electronic devices.

The sterile controller could also be used in other environments where a sterile or clean input device would provide benefits. For example, the sterile controller could be used in clean rooms, pathology labs and food processing plants.

Although the description above contains many specificities, these should not be construed as limiting the scope of the embodiments but as merely providing illustrations of some of the several embodiments. Thus the scope of the embodiments should be determined by the appended claims and their legal equivalents, rather than by the examples given.

We claim:

1. An input device, comprising:
a sterile faceplate, including at least one sterile physical control mounted to said faceplate; and
a housing containing a removable, autoclavable sensing module, wherein said sensing module detects said physical control when said faceplate is fastened to said housing.

2. The input device of claim 1, wherein said sensing module is rechargeable.

3. The input device of claim 1, wherein said sensing module can be inductively recharged.

4. The input device of claim 1, wherein said sensing module includes thermal insulating material.

5. The input device of claim 1, wherein a partial vacuum is created within said sensing module.

6. The input device of claim 1, wherein said housing is sterile.

7. The input device of claim 1, wherein said sensing module uses a camera to detect said physical control.

8. The input device of claim 1, wherein said sensing module uses a capacitive sensor to detect said physical control.

9. A method for controlling computing devices or medical equipment in a sterile or clean environment, comprising the steps of:
providing a sterile housing;
providing a sterile faceplate, including at least one sterile physical control mounted to said faceplate;
providing an autoclavable sensing module;
inserting said sensing module into said housing;
fastening said faceplate to said housing;
using said sensing module to sense said physical control when said physical control is manipulated by a user.

10. A method according to claim 9, further comprising the step of disposing said housing, said faceplate and said physical control after a procedure.

11. A method according to claim 9, further comprising the step of re-sterilizing said housing, said faceplate and said physical control after a procedure.

12. A method according to claim 9, further comprising the step of autoclaving said sensing module after a procedure.

13. A method according to claim 9, further comprising the step of recharging said sensing module after a procedure.

14. A method according to claim 9, further comprising the step of recharging said sensing module after a procedure and wherein said sensing module is recharged using an inductive charger.

15. An input device, comprising:
a sterile faceplate, including at least one sterile physical control mounted to said faceplate; and
an autoclavable housing containing non-removable electronics capable of detecting said physical control when said faceplate is fastened to said autoclavable housing.

16. Apparatus according to claim 15, wherein said faceplate can be autoclaved.

17. Apparatus according to claim 15, wherein said housing is rechargeable.

18. Apparatus according to claim 15, wherein said housing can be inductively charged.

19. Apparatus according to claim 15, wherein said housing uses a camera to detect said physical control.

20. Apparatus according to claim 15, wherein said housing uses a capacitive sensor to detect said physical control.
